(19) United States
(12) Patent Application Publication
(10) Pub. No.: US 2016/0213417 A1
(43) Pub. Date: Jul. 28, 2016

(54) DEVICE FOR OBLITERATING TISSUE

(71) Applicant: JOIMAX GMBH, Karlsruhe (DE)

(72) Inventor: Wolfgang RIES, Linkenheim (DE)

(21) Appl. No.: 14/914,475

(22) PCT Filed: Aug. 25, 2014
(86) PCT No.: PCT/EP2014/002316

§ 371 Date: Feb. 25, 2016

(30) Foreign Application Priority Data
Aug. 29, 2013 (DE) 20 2013 007 654.2

(51) Int. Cl. A61B 18/14

(52) U.S. Cl. CPC A61B 18/14; A61B 2018/1475; A61B 2018/00434

(57) ABSTRACT

A device for obliterating tissue with a bipolar probe, which has a distal end part with two electrodes electrically insulated against one another by an insulating web which is electrically insulating. To make such a device with a bipolar probe safer, the electrodes have depressions located opposite each other, which are filled by electrically insulating insulating material.

### CROSS REFERENCE TO RELATED APPLICATIONS

This application is a United States National Phase Application of International Application PCT/EP2014/002316 filed Aug. 25, 2014 and claims the benefit of priority under 35 U.S.C. §119 of German Application 20 2013 007 654.2 filed Aug. 29, 2013 the entire contents of which are incorporated herein by reference.

### FIELD OF THE INVENTION

The present invention pertains to a device for obliterating tissue with a bipolar probe, which has a distal end part with two electrodes insulated electrically against each other by an electrically insulating insulating web.

### BACKGROUND OF THE INVENTION

Tissue is obliterated in the body, for example, denervation, such as facet joint denervation is also performed by means of such probes. A bipolar probe has two electrodes, via which a voltage difference is applied. To enable the distal end of the probe to be able to be inserted flexibly and to also enable areas located laterally from the axial direction of the main body of the probe to be reached, the probe has an elastically flexible distal end area. On the other hand, the probe is inserted into the body such that it is pulled extensively into a shaft connected to grip parts, in which shaft the distal end area of the probe is pulled into the stretched position against the prestress.

Because of the motions of the distal end area of the probe between stretching and bent position and prestressed bent position, axial forces are applied to the connection between the electrodes and the electrically insulating separating layer connected to them, while, as a result of which detachment may occur. This is disadvantageous.

### SUMMARY OF THE INVENTION

A basic object of the present invention is therefore to make a device of this class with a bipolar probe safer.

This object is accomplished according to the present invention in a device of this class by the electrodes having mutually opposite depressions, which are filled by electrically insulating insulating material. Provisions may be made in this connection in alternative preferred embodiments for the recesses to be designed as partly cylindrical and for the insulating layer to have a cylindrical expansion or for the recesses to be designed as partly spherical and for the insulating layer to have a spherical expansion.

Provisions are made in other embodiments of the present invention for the electrodes to be rigidly connected to the insulating web, the electrodes being connected to the insulating web, in particular, in substance, and the insulating web being formed by injection or pouring in between the electrodes, or the electrodes being bonded to the insulating web.

Provisions are made in the first case, in particular, for the insulating material to be formed by thickened parts of the insulating web, which protrude into the depressions of the electrodes, wherein the electrodes are connected to one another and to the insulating web, in particular, by injection or pouring in insulating web and by expansions thereof.

When bonding insulating layers and electrodes, provisions may be made either for the insulating material to be a cured adhesive filling the depressions and rigidly connecting the insulating layer and the electrodes or for the insulating material to be formed by thickened parts of the insulating layer protruding into the depressions of the electrodes. The latter can likewise be brought about during the preparation of the insulating layer between the electrodes by injection or pouring. In case of bonding the electrodes and insulting layer, provisions may, furthermore, be made for the insulating layer likewise to have, in the area of the depressions of the electrodes, depressions directed on both sides towards the electrodes, which said depressions are filled with cured adhesive, so that a positive-locking connection and a connection in substance will then also be given with the insulating layer.

Provisions are made in another preferred embodiment for the distal end part of the probe to be connected proximally to a flexible tube, wherein the flexible tube part is held over its greatest length in a rigid metallic pipe and only the distal end area of the flexible tube part protrudes from this displaceably. The connection between the electrodes and the flexible tube part is likewise a rigid connection, preferably a connection in substance, which is formed especially by bonding.

Provisions are made in another embodiment for the distal end area of the flexible tube part to be prestressed in a bent state, wherein the flexible tube has, furthermore, two ducts (lumens), separated by a partition.

To apply electric current to the distal electrodes from the proximal end of the probe, provisions are made in another preferred embodiment for the electrodes to be connected electrically conductively to proximal terminal contacts via electric conductors and for the probe to have a tube jointing sleeve with a radial incision in the proximal area, by means of which incision the probe can be fixed axially and angularly in a grip. The material of the flexible tube part is preferably an elastomer. The connection wires from the proximal contacts to the distal electrodes are stripped of the insulation at their ends.

Provisions are made, furthermore, in an extremely preferred embodiment for the distal end of the probe to be able to be pushed distally out of a shaft connected to one of the grip parts by moving two grip parts of the grip against one another.

Further advantages and features of the present invention appear from the claims and from the following description, in which an exemplary embodiment of the present invention is explained in detail with reference to the drawings.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a view showing a probe according to the present invention in a side view;

FIG. 2 is a view showing an enlarged longitudinal sectional view of the distal area of the probe;

FIG. 3 is a cross sectional view through the distal area of the probe at the section III-III indicated in FIG. 2;

FIG. 4 is a cross sectional view through the distal area of the probe at the section IV-IV indicated in FIG. 2;

FIG. 5 is a cross sectional view through the distal area of the probe at the section V-V indicated in FIG. 2;

FIG. 6 is a view showing an enlarged longitudinal sectional view of the proximal area of the probe from FIG. 1;

FIG. 7 is a view showing a probe inserted in an actuating grip in a non-actuated position according to FIG. 1, partly in a longitudinal section; and

FIG. 8 is a view showing a view corresponding to FIG. 7 in the actuated position of the grip.

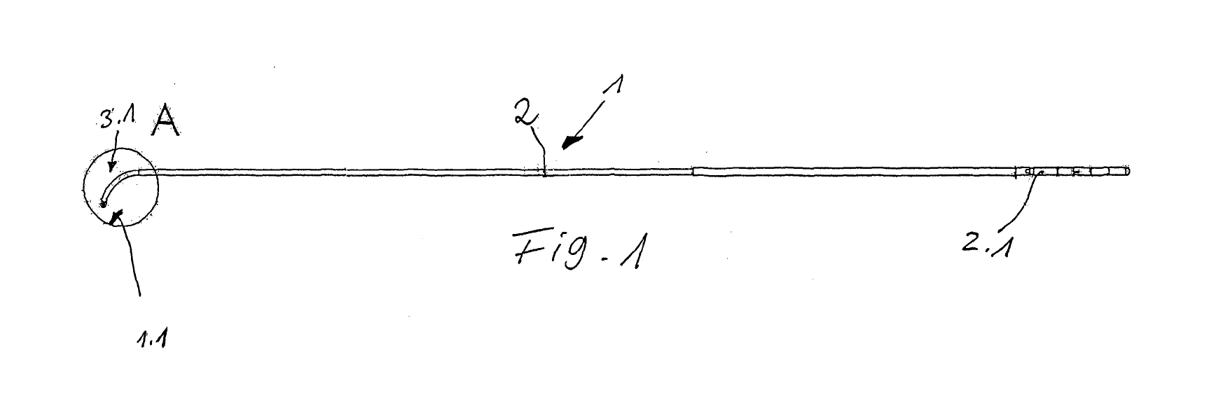

### DESCRIPTION OF THE PREFERRED EMBODIMENTS

The probe 1 according to the present invention has an outer metal pipe 2, through which a double-lumen plastic flexible tube 3 is led, which protrudes at the distal end of the pipe 2 with a bent, prestressed distal end 3.1. The flexible tube 3 has an outer wall 3.2 with a circular cross section and two ducts, which are insulated by a partition 3.3 and have a partly circular cross section.

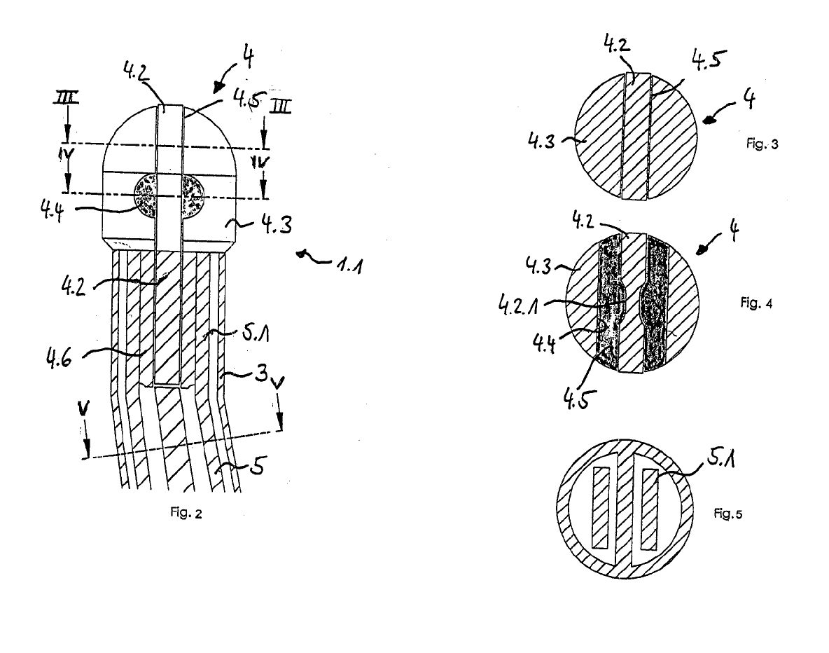

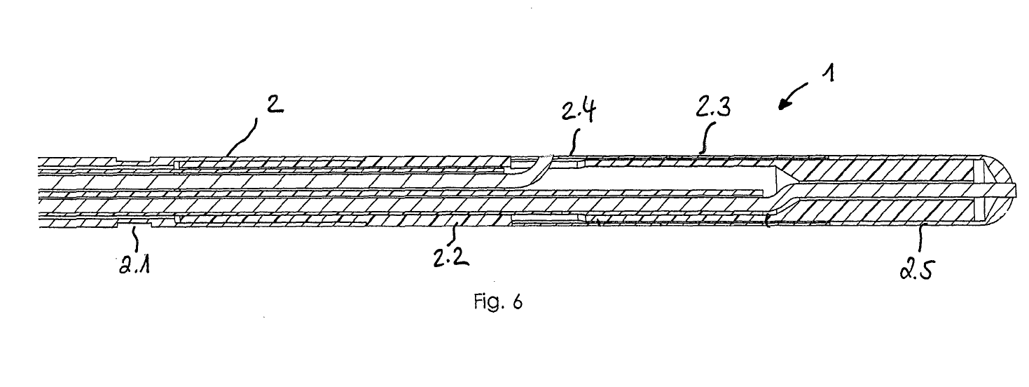

A probe head 4 (FIG. 2), which forms a distal end part 1.1 of the probe 1 and is preferably connected to the flexible tube 3 in substance, especially by bonding (e.g., with silicone adhesive) in the exemplary embodiment shown in FIG. 4, is inserted into the distal end 3.1 of the flexible tube 3.

The probe head 4 has a partly spherically rounded end face 4.1. The probe head 4 has, furthermore, two electrodes 4.3 separated from one another by an insulating web 4.2 consisting of plastic in the form of metal bodies. The electrodes 4.3 are designed at first as partly cylindrical in their end facing away from the end face 4.1. Their distal end is such that the distal end face of the probe head as a whole is partly spherical, especially hemispherical. The electrodes 4.3 have, in their surfaces facing each other, transversely extending, partly cylindrical depressions 4.4. The depressions extend at right angles to the insertion direction parallel to one another. They are not flush in the direction in which they extend, but have a finite distance at right angles to this. The depressions 4.4 are filled here by cured plastic 4.5 connecting in substance a cylindrical expansion 4.5.1 of the plastic insulating web 4.2 and the electrodes 4.3.

Instead of partly cylindrical depressions extending in the transverse direction and a corresponding cylindrical expansion 4.5.1, partly spherical depressions in the electrodes 4.3 and a corresponding spherical thickened part of the adhesive 4.5 may be provided. Further, there are depressions 4.2.1 in the insulating web 4.2, which are likewise filled by cured adhesive 4.5. In addition to the connection of the electrodes 4.3 and the intermediate layer 4.2 in substance, a positive-locking connection may also be established by the expansion 4.5.1 of the cured adhesive 4.5.

Instead of by bonding, the connection in substance of the electrodes 4.3 and the insulating layer 4.2 may also be established by injecting or pouring in the insulating layer between the electrodes 4.3.

Due to this design especially with the depressions 4.4 and the expansions 4.5.1, any axial displacement of the electrodes 4.3 during the bending of the distal end 3.1 of the flexible tube 3 is avoided when the curvature of said flexible tube is changed, due, for example, to pushing into and out of another outer pipe (see below) or due to pressing against tissue.

The electrodes 4.3 of the head 4 have proximal plate-like attachments 4.6. The head 4 is connected in substance, especially by bonding, on the one hand, at the distal end-face end of the flexible tube 3 to the outer wall 3.2 of said end and, on the other hand, to distal lateral surfaces of the partition 3.3 via the attachments 4.6. Furthermore, stripped ends of connection wires 5 are fixed on the outer sides of the plates 4.6 by connection in substance, for example, by soldering or also by bonding with an electrically conductive adhesive. Electric voltage can be applied from the proximal end of the probe 1 via the wires 5 on the partial body acting as electrodes 4.3.

The connection wires are designed as round wires over their length and are preferably insulated, for example, by insulating lacquer or else a usual outer plastic flexible tube (not shown in detail). In their distal area 5.1, they are flattened in the above-described manner for connection to the electrodes 4.3.

At its proximal end area, the metal pipe 2 has a radial incision 2.1, via which the probe 1 can be fixed axially and angularly in a grip. The probe 1 has, furthermore, at first a contact insulation 2.7 separating the pipe 3 from a contact socket 2.4 in the proximal direction and, adjoining the contact socket 2.4, an insulation piece 2.3 separating this from a proximal contract bush 2.5. The contact socket 2.4 and the contact bush 2.5 are used as terminal contacts. One of the connection wires 5 is connected electrically conductively to the socket 2.4 and the connection wire is connected electrically conductively to the bush 2.5, and the latter wire being guided with a stripped end in this bush, which closes off the probe 1 on the proximal end face and is in electrically conductive connection with the bush 2.5. The parts are connected by bonding.

All metallic parts, especially electrodes 4.3, pipe 3, contact socket and contact bush 2.4, 2.5 and connection wires 5 preferably consist of special steel. The electrically conductive parts consist of plastic and the double-lumen flexible tube 3 consists, in particular, of an elastomer, such as polyether block amide (PEBA; commercially available, for example, under the trade name PEBAX), the insulating web 4.2 between the electrodes consists of polyamide (PA); the adhesive 4.5 connecting the latter is an epoxy resin adhesive; the insulation piece 2.3 consists of polyoxymethylene (POM).

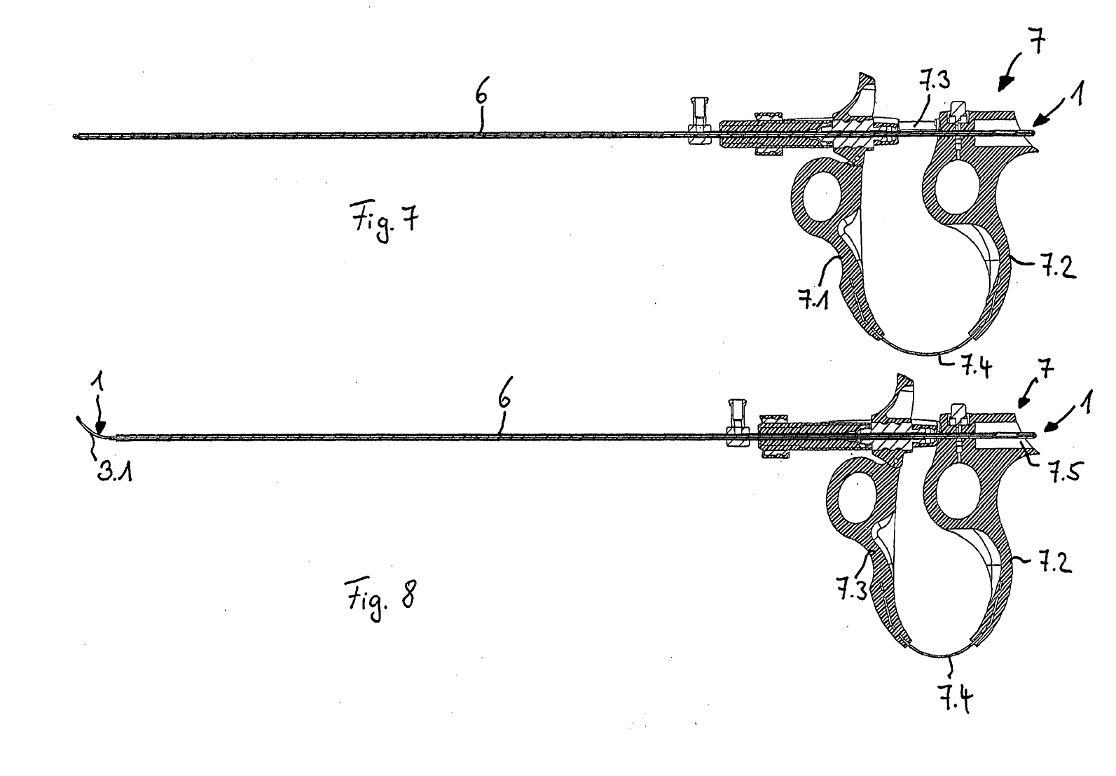

For use, the probe 1 is inserted into a grip 7 having a metal shaft 6. The grip 7 comprises two grip parts 7.1, 7.2, which are elastically connected to one another and which are displaceable in relation to one another via a guide pin 7.3. The probe 1 is fixed axially and angularly via the radial incision 2.1. The grip parts 7.1, 7.2 are kept at a distance in the inoperative position in relation to one another via an elastic spring-loaded connection part 7.4, i.e., they are kept at a distance from one another without load, as this is shown in FIG. 7. The distal end of the probe 1 is moved extensively into the shaft 6 and projects over same only slightly, as this is shown in FIG. 7. By actuating the grip 7 by moving the grip parts 7.1, 7.2 in relation to one another along the guide pin 7.3 against the spring action of the connection part 7.4, the probe is moved through the shaft 6 in the distal direction by means of the grip part 7.2, so that the distal tip 3.1 of the probe 1 is moved distally out of the shaft 6 and it can assume its prestressed curvature (FIG. 8), while it is held stretched in the withdrawn position according to FIG. 7 (with the grip not stressed) by the shaft 6.

Towards the proximal end of the probe 1, the grip part 7.2 has a coaxial recess 7.5, into which a connector plug with electric contacts can be inserted for contacting the metal sockets 2.4, 2.5.

As was stated, the distal end 3.1 of the probe 1 is moved out of the shaft 6 by actuating the grip 7 and can contact tissue. When voltage is applied to the probe 1 via the described electrically conductive parts, which are connected to one another, obliteration of tissue, etc., can then be performed.

While specific embodiments of the invention have been shown and described in detail to illustrate the application of the principles of the invention, it will be understood that the invention may be embodied otherwise without departing from such principles.

1. A device for obliterating tissue with a bipolar probe, the device comprising:
a distal end part with two electrodes;
an insulating web that is electrically insulating, the two electrodes being electrically insulated against one another by the insulating web, wherein the electrodes have depressions located opposite each other; and
electrically insulating insulating material filling the depressions of the electrodes.

2. A device in accordance with claim 1, wherein the depressions comprise recesses that are partly cylindrical; and the insulating material has a cylindrical expansion.

3. A device in accordance with claim 2, wherein the partly cylindrical recesses do not extend flush but at a finite distance in parallel to one another.

4. A device in accordance with claim 1, wherein the depressions comprise recesses that are partly spherical and the insulating material filling the depressions has corresponding partly spherical expansions.

5. A device in accordance with claim 1, wherein the electrodes are rigidly connected to the insulating web.

6. A device in accordance with claim 1, wherein the insulating material filling the depressions is the same material as that of the insulating web.

7. A device in accordance with claim 1, wherein the electrodes are connected to the insulating web in substance.

8. A device in accordance with claim 1, wherein the insulating layer is formed by injecting or pouring in between the electrodes.

9. A device in accordance with claim 1, wherein the insulating material is formed by thickened parts of the insulating web protruding into the depressions of the electrodes.

10. A device in accordance with claim 8, wherein the electrodes are connected to one another and to the insulating web by injecting or pouring in insulating web and expansions thereof.

11. A device in accordance with claim 1, wherein the electrodes are bonded to the insulating web by means of plastic.

12. A device in accordance with claim 1, wherein the insulating material is a cured adhesive, which fills out the depressions and rigidly connects the insulating web and the electrodes.

13. A device in accordance with claim 1, wherein in the area of the depressions of the electrodes, the insulating web is a layer that has depressions on both sides thereof and extending in a direction towards the electrodes and which are filled with cured adhesive.

14. A device in accordance with claim 1, further comprising a flexible tube, wherein the distal end part of the probe is connected proximally to the flexible tube.

15. A device in accordance with claim 14, wherein the distal end area of the flexible tube is prestressed in a bent state.

16. A device in accordance with claim 14, wherein the flexible tube has two ducts (lumens), separated by a partition.

17. A device in accordance with claim 1, further comprising electric conductors with proximal terminal contacts, wherein the electrodes are connected to one another electrically conductively via the electric conductors with proximal terminal contacts.

18. A device in accordance with claim 1, wherein the probe further comprises a tube jointing sleeve with a radial incision in a proximal area, by means of which incision the probe can be fixed axially and angularly in a grip.

19. A device in accordance with claim 18, wherein the distal end of the probe can be pushed out of a shaft connected to one of the grip parts by moving two grip parts of the grip against one another.
